(19) United States
(12) Patent Application Publication
(10) Pub. No.: US 2024/0428402 A1
(43) Pub. Date: Dec. 26, 2024

(54) SYSTEM AND METHOD FOR PREDICTION OF OBSTRUCTIVE CORONARY ARTERY DISEASE

(71) Applicants: Far Eastern Memorial Hospital, New Taipei City (TW); National Taiwan University, Taipei (TW); National Taiwan University Hospital, Taipei City (TW)

(72) Inventors: Yen-Wen WU, Taipei (TW); Chi-Lun KO, Taipei (TW); Chung-Ming CHEN, Taipei (TW)

(21) Appl. No.: 18/341,316

(22) Filed: Jun. 26, 2023

(51) Int. Cl. G06T 7/00; G06T 3/00; G06T 3/40; G06T 7/11; G06T 7/30; G06T 15/00; G16H 50/20

(52) U.S. Cl. CPC G06T 7/0012; G06T 3/06; G06T 3/40; G06T 7/11; G06T 7/30; G06T 15/00; G16H 50/20; G06T 2207/20084; G06T 2207/30048

(57) ABSTRACT

Provided is a system and method for prediction of obstructive coronary artery diseases, where a pre-processing module is configured to generate a left ventricular myocardium image from 3D images of a subject that is space-invariant, a flattening module is configured to resample the left ventricular myocardium image into flattened image in 3D spherical coordinate and preserve neighborhood relationship between myocardium of the subject, and a deep learning module is configured to predict probabilities of obstructive coronary artery disease in left anterior descending, left circumflex and right coronary artery and probability of patent coronary artery for calculation of compound probability of obstructive coronary artery disease for the subject. Therefore, the present disclosure may achieve full automation and take advantage of 3D information in prediction of obstructive coronary artery disease via MPI, thus does not require polar maps, manual correction or NDB derived quantification for prediction, thereby outperform traditional TPD quantification in prediction of obstructive CAD.

### TECHNICAL FIELD

The present disclosure relates to medical imaging technology, more particular to a system and a method for prediction of obstructive coronary artery disease.

### DESCRIPTION OF RELATED ART

Myocardial perfusion imaging (MPI) has been widely applied in diagnosis of coronary artery disease (CAD) and in assessment of cardiovascular risk. MPI provides valuable functional information and serves as a gatekeeper of invasive coronary angiography (ICA) and coronary interventions. For example, when MPI showed significant ischemia, it is implied that revascularization should outperform medical treatment in reducing ischemic burden and will provide survival benefit. Moreover, MPI is also important in following-up CAD patients and providing prognostic implications.

Cardiac CZT cameras are beneficial for MPI since they are capable of reducing MPI acquisition time while preserving diagnostic accuracy. Gated MPI can be routinely acquired and provide valuable information. However, specially designed collimation system for cardiac CZT cameras often cause different attenuation patterns in MPIs compared to those in conventional single-photon emission computed tomography (SPECT), meaning previously established normal databases (NDB) for conventional SPECT may no longer be used, and efforts will be required to create new NDBs to account for the changes.

Machine learning (ML) have been largely applied to the analysis of medical images in recent years. In contrast to handcrafted algorithms that are designed based on human experience, ML models learn directly from data. ML models allow incorporation of biomedical information, such as demographic data and radiomic features, to make prediction. In the realm of machine learning techniques for this matter, deep learning (DL) enables models to take image as input directly and to capture image features or patterns by themself.

Polar map (PM) based analysis has also been a popular mean in clinical interpretation in analysis of MPI, where PM or PM-derived parameters can provide means to combine or compare different myocardial images in a same 2D coordinate system and allow presentation of holistic 3D MPI or quantitative data in approachable 2D form. A few quantification parameters, such as commonly used total perfusion deficit (TPD), are derived with PM-based analysis. However, PM-based analysis often requires a predefined model, such as ellipsoid surface or hemisphere plus cylinder, to transform 3D myocardium into 2D PM, meaning the selection of basal valve plane will be problematic and will largely influence the quantitative results. Further, transformation from 3D myocardium into 2D PM also contains a process of data condensing, either by taking the maximum circumferential count profile, or by averaging myocardial voxel activity perpendicular to a fitting surface, which will result in information loss therebetween.

A few previously published studies have applied ML or DL to MPI, and various deep learning-based models (DL models) has been shown to outperform traditional quantification (such as traditional TPD quantification) in predicting obstructive CAD from MPI by using quantitative PM. They usually start from MPI automatic quantification and NDB comparison as in standard QPS (quality positioning services) software (such as the ones developed by Cedars-Sinai Medical Center), and quantitative PMs resulted from said comparison are then fed into the DL models to predict obstructive CAD. Although PM is useful in quantification of MPI, the problem of imperfect automatic ellipsoid fitting in PMs using DL models is still present, which may result in erroneous left ventricular (LV) myocardial contouring. To be more specific, manual correction in 12% to 21% of MPI images are still required for current DL models.

Therefore, there is an unmet need to achieve full automation and take advantage of 3D information in prediction of obstructive coronary artery disease via MPI.

### SUMMARY

In view of the foregoing, the present disclosure provides a system and a method for prediction of obstructive coronary artery disease, the system and the method is configured to configuring a pre-processing module to pre-process a MPI image set of a subject in need thereof into left ventricular myocardium images of post-stress form and rest form, wherein the MPI image set comprises 3D images of post-stress form and rest form: configuring a flattening module to resample the left ventricular myocardium images into flattened images of post-stress form and rest form, wherein the flattened images comprises data in 3D spherical coordinate system; and configuring a deep learning module to take the left ventricular myocardium images and the flattened images as input for prediction of obstructive coronary artery disease for the subject. Also provided is a method for processing the MPI image of the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following descriptions of the embodiments, with reference made to the accompanying drawings, wherein:

FIG. 1 is a schematic diagram illustrating system for prediction of obstructive coronary artery disease in accordance with embodiments of the present disclosure;

FIG. 2 is a schematic flow chart illustrating method for prediction of obstructive coronary artery disease in accordance with embodiments of the present disclosure;

FIG. 3 is a schematic diagram illustrating structure of a 3D U-net for segmenting LV myocardium from 3D images in accordance with embodiments of the present disclosure:

FIG. 4 is a schematic diagram illustrating process for generation of 3D blackout images in accordance of the present disclosure:

FIGS. 5A and 5B are schematic diagrams illustrating structures of deep learning models for prediction of obstructive coronary artery disease in accordance with embodiments of the present disclosure; and

FIG. 6 illustrates patient-based analysis of the performance of the system and method for prediction of obstructive coronary artery disease in accordance with embodiments of the present disclosure.

FIG. 7 illustrates vessel-based analysis of the performance of the system and method for prediction of obstructive coronary artery disease in accordance with embodiments of the present disclosure.

FIG. 8A illustrates one representative case of vessel-based analysis of the performance of the system and method for prediction of obstructive coronary artery disease in accordance with embodiments of the present disclosure. A 66-year-old man with hypertension and dyslipidemia. Three vessel disease was confirmed by ICA.

FIG. 8B illustrates the other representative case of vessel-based analysis of the performance of the system and method for prediction of obstructive coronary artery disease in accordance with embodiments of the present disclosure. A 61-year-old woman with hypertension, dyslipidemia, and patent coronary arteries.

### DETAILED DESCRIPTION OF THE EMBODIMENTS

The following specific embodiments are provided to illustrate the disclosure of the present disclosure in detail, a person having ordinary skill in the art can easily understand the advantages and effects of the present disclosure after reading the disclosure of this specification, and also can implement or apply in other different specific embodiments. Therefore, any element or method within the scope of the present disclosure disclosed herein can combine with any other element or method disclosed in any specific embodiment of the present disclosure.

The proportional relationships, structures, sizes and other features shown in accompanied drawings of this disclosure are only used to illustrate embodiments describe herein, such that those with ordinary skills in the art can read and understand the present invention therefrom, of which are not intended to limit the scope of this disclosure. Any changes, modifications, or adjustments of said features, without affecting the designed purposes and effects of the present disclosure, should all fall within the scope of technical content of this disclosure.

As used in this specification, when describing an object “comprises,” “includes” or “has” a specification, unless otherwise specified, it may additionally include other elements, components, structures, regions, parts, devices, systems, steps, connections, etc., and should not exclude other specifications.

As used in this specification, sequential terms such as “first,” “second,” etc., are only cited in convenience of describing or distinguishing specifications such as elements, components, structures, regions, parts, devices, systems, etc. from one another, which are not intended to limit the application scope of this disclosure, nor to limit spatial sequences between such specifications. Further, unless otherwise specified, wordings in singular forms such as “a” and “the” also pertain to plural forms, and wordings such as “or” and “and/or” may be used interchangeably.

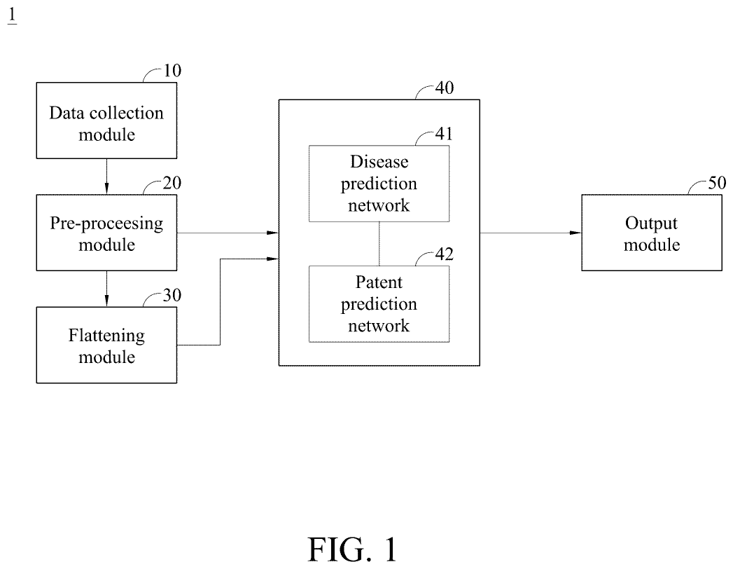

Referring to FIG. 1, a system 1 for prediction of obstructive coronary artery disease is disclosed, comprising: a data collection module 10, a pre-processing module 20, a flattening module 30, a deep learning module 40, and an output module 50. Further, the arrows denoted between elements of said system 1 indicates the operating relationships therebetween, which will be detailed later. Moreover, described elements of the system 1 may be connected to each other via any suitable wired or wireless means, of which the present disclosure is not limited thereto.

In some embodiments, the data collection module 10 is configured to collect a MPI image set from arbitrary subject (patient) underwent MPI (e.g., thallium-201 MPI) for prediction of obstructive coronary artery disease. In practice, the MPI image set is comprised of 3D images imagining at least left ventricular (LV) myocardium of the subject in a post-stress form and a rest form, respectively. Further, the data collection module 10 may be coupled to or implemented with a data source that provides or stores the MPI image sets, such as a CZT camera, a camera system, a data storing device, system, database, cloud storage, or the like, of which the present disclosure is not limited thereto.

In some embodiments, the pre-processing module 20 is configured to automatically pre-process the MPI image sets received by the data collection module 10. In practice, the automatic pre-process of the MPI image set performed by the pre-processing module 20 may comprise at least the process of: performing a left ventricular (LV) myocardial segmentation on the 3D images of the MPI image set to obtain LV myocardium images corresponding to the 3D images: performing a rigid registration to align LV myocardium images to a predetermined myocardium template; and performing a normalization to normalize maximum voxel value of each LV myocardium image to mean of the greatest 20% voxel value of the segmented LV myocardial. It should be noted that the pre-processing process performed by the pre-processing module 20 is not limited those mentioned above, and can include other applicable processes that are beneficial for prediction result in a later stage, of which the present disclosure is not limited thereto.

In some embodiments, the flattening module 30 is configured to resample LV myocardium images of the MPI image set pre-processed by the pre-processing module 20 into flattened images so as to preserve neighborhood relationship between myocardium. In practice, the flattened image is generated by resampling the LV myocardium image from Cartesian coordinate system into 3D spherical coordinate.

In some embodiments, the deep learning module 40 is configured to take the LV myocardium images of the MPI image set pre-processed by the pre-processing module 20 and the flattened images corresponding to the LV myocardium images output from the flattening module 30 into consideration for prediction of obstructive CAD for the subject. In practice, the deep learning module 40 is comprised of a disease prediction network 41 and a patent prediction network 42, where the disease prediction network 41 is configured to predict probability of obstructive CAD in left anterior descending (LAD), left circumflex (LCX) and right coronary artery (RCA) for the subject based on the LV myocardium images and the flattened images, respectively, and the patent prediction network 42 configured to predict probability of patent coronary arteries for the subject. The disease prediction network 41 and the patent prediction network 42 are preferably realized as convolution neural networks (CNN), but other types of deep learning network may also be applied, of which the present disclosure is not limited thereto. The deep learning module 40 may also have other configurations. For example, the disease prediction network 41 may be realized as three separate deep learning networks configured to calculate probability of obstructive CAD in LAD, LCX and RCA for the subject, respectively, or the disease prediction network 41 and patent prediction network 42 may also instead be realized as a single deep learning network to calculate probability of obstructive CAD in LAD, LCX and RCA and probability of patent coronary arteries for the subject at the same time, of which the present disclosure is not limited thereto.

In some embodiments, the output module 50 is configured to provide a compound probability of obstructive CAD of the subject based on the predictions made by the deep learning module 40. In practice, the output module 50 may be a display that configured to present the compound probability in numeral forms for further analysis by cardiologists. However, the output module 50 may be realized in other forms, and the mean of presenting the compound probability may also be realized in other means, of which the present disclosure is not limited thereto.

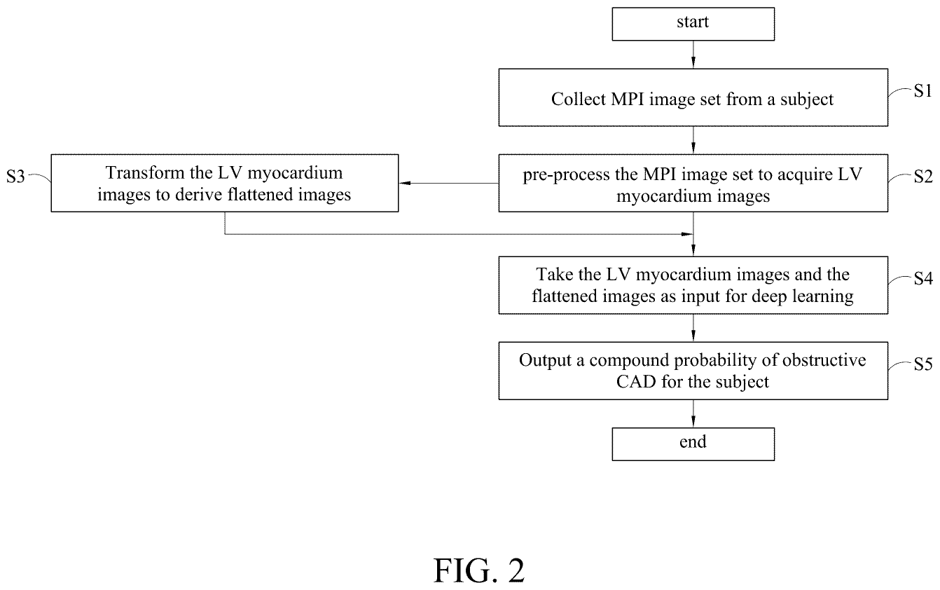

Referring to FIG. 2, steps for prediction of obstructive coronary artery disease via operation of the system 1 of FIG. 1 is disclosed and explained herefrom.
At step S1, a MPI image set of a subject is collected by the data collection module 10, where the MPI image set comprises 3D images in a post-stress form and a rest form collectively respectively.
At step S2, the 3D images of the MPI image set is pre-processed by the pre-processing module 20 into LV myocardium images (in post-stress form and rest form corresponding to the 3D images) in automatic fashion, where the pre-processing of the 3D images comprises performing a LV myocardial segmentation on the 3D images to obtain LV myocardium images corresponding to the 3D images: performing a rigid registration to align LV myocardium images to a predetermined myocardium template; and performing a normalization to normalize maximum voxel value of each LV myocardium image to mean of the greatest 20% voxel value of the segmented LV myocardial.
At step S3, the LV myocardium images are resampled from Cartesian coordinate system into 3D spherical coordinate by the flattening module 30 to obtain flattened images (also in post-stress form and rest form corresponding to the 3D images) corresponding to the LV myocardium images.
At step S4, the deep learning module 40 takes the LV myocardium images and the flattened images in both post-stress form and rest form to calculate probability probabilities of obstructive CAD in LAD, LCX and RCA for the subject, respectively, and takes the LV myocardium image in post-stress form to calculate the probability of patent coronary arteries for the subject.
At step S5, the probabilities of obstructive CAD in LAD, LCX and RCA and the probability of patent coronary arteries are calculated as a compound probability by the output module 50 to indicate the probability of obstructive CAD for the subject.

From here, a detailed description of how working mechanisms of the data collection module 10, the pre-processing module 20, the flattening module 30, the deep learning module 40, and the output module 50 are designed will be provided.

### Material and Methods

### Study Population

For the sake of development of the system 1 of the present disclosure, 1861 consecutive subjects (patients) referred for SPECT MPI during a span of five years are enrolled for study using CZT cameras at National Taiwan University Hospital. All subjects have neither history of percutaneous coronary intervention, coronary bypass surgery, nor myocardial infarction, and all of them have received invasive coronary angiography (ICA) within 90 days of MPI based on clinical indication. It is noted that the perfusion tracer used in MPI for the subject enrolled is preferably thallium-201, but the present disclosure is not limited thereto. It is further noted that CZT cameras used in MPI for the subject enrolled may include one or more types, which the present disclosure is also not limited thereto.

The enrolled subjects are further divided into internal parameterization group (i.e., subjects underwent MPI before a middle timestamp of said five years, n=928) and external validation group (i.e., subjects underwent MPI after the middle timestamp of said five years, n=933). The data in internal parameterization group was used to develop and to train the prediction model, while the external validation group is kept away from all model developing and training processes of the system 1 of the present disclosure. It mimicked patients whom the developed model may encounter after future deployment. The partition of the internal parameterization group and the external validation group is not randomized (i.e., divided based on continuous timeline of enrollment), meaning there will be significant differences between their baseline characteristics, and may be useful for validation of development of the system 1 of the present disclosure.

In addition, another group of 30 subjects having documented patent coronary arteries and nearly normal MPI are also collected for building of a normal database (NDB). The NDB is built to perform conventional TPD quantification, to generate black-out map, and to test whether NDB-based quantification was needed by the proposed model for development of the system 1 of the present disclosure before putting into practical use.

### Stress Test and Image Acquisition

In the embodiments described herein, MPI image sets of the enrolled subjects are collected by the data collection module 10 for developing (i.e., MPI image sets related to the internal parameterization group) and validating (i.e., MPI image sets related to the external validation group) of the system 1 of the present disclosure.

During study, all enrolled subjects underwent either standardized dipyridamole pharmacologic stress test (n=1436, about 77% of total subjects) or treadmill exercise with Bruce protocol (n=425, about 23% of total subjects). Post-stress images are required within 4 to 6 minutes after intravenous injection of 111 MBq (3mCi) thallium-201 at peak stress using a CZT SPECT camera (such as a Discovery NM530c developed by GE Healthcare, Haifa, Israel) and collected by the data collection module 10. After resting for at least 4 hours, rest images (also known as redistribution images in the case of thallium-201-related MPI, but the present disclosure is not limited thereto) are required on the same camera and accordingly collected by the data collection module 10 thereafter. In the embodiments described herein, acquisition time is 3 to 4 minutes for the post-stress images and 4 to 6 minutes for the rest images. The post-stress images and the rest images are then reconstructed by the data collection module 10 with default settings and is anonymized with other clinical data. The reconstructed post-stress images and redistribution images are then exported by the data collection module 10 as 3D arrays of 32-bit floating point values (70×70×70 voxels) for further analysis (collectively referred as 3D images, which are differentiated in post-stress form and rest form). The exported 3D images are further cropped by the data collection module 10 into a 64×64×64 matrix at their center to reduce empty boundaries, hence forming the MPI image set for the subjects.

### Invasive Coronary Angiography

ICA is performed based on clinical routines. All ICAs are performed and visually interpreted by cardiologists. A 50% or more luminal stenosis of left main artery or a 70% or more luminal stenosis of other three epicardial vessels will be considered as obstructive disease. Subjects with obstructive left main disease are considered to have both left anterior descending (LAD) and left circumflex (LCX) diseases. During study, vessels status observed from ICA is served as reference standard, which will be utilized during validation of the system of the present disclosure.

### Creation of Myocardium Template

In the embodiments described herein, 145 visually normal redistribution 3D SPECT images, whose summed rest score (SRS) less than 4, from the internal parameterization group are manually selected for creation of myocardium template. Then, 1 of the 145 selected redistribution images with most centrally located myocardium is chosen to serve as a target. Next, a rigid transformation is performed to align the rest of the 144 selected redistribution images to said target. Finally, the myocardium template is created by averaging the 145 selected redistribution images.

The process for creation of the myocardium template as described above is not meant to limit the scope of the present disclosure and can be conducted via various means. For example, the creation of the myocardium template may be performed on the pre-processing module 20 (e.g., having the pre-processing module 20 provide an interface to operate the process described above) at real time, or the myocardium template may be stored in the pre-processing module 20 after being created by outside resources, of which the present disclosure is not limited thereto. Additionally, the myocardium template may further be iteratively improved by the pre-processing module 20 following continuous collection of MPI image sets from subjects during practical use of the system 1 of the present disclosure, of which the present disclosure is also not limited thereto.

### Automatic Data Preprocess

In the embodiments described herein, automatic data preprocess of MPI image sets is realized by the pre-processing module 20 in three processes. Firstly, a left ventricular (LV) myocardium segmentation is performed on the 3D images of the MPI image sets by using a well-trained U-net model to obtain LV myocardium images therefrom. Then, a rigid registration is performed to align each LV myocardium image to the myocardium template according to the segmented LV myocardium thereof. Finally, a normalization is performed to normalize maximum voxel value of each LV myocardium image to mean of the greatest 20% voxel value of the segmented LV myocardial.

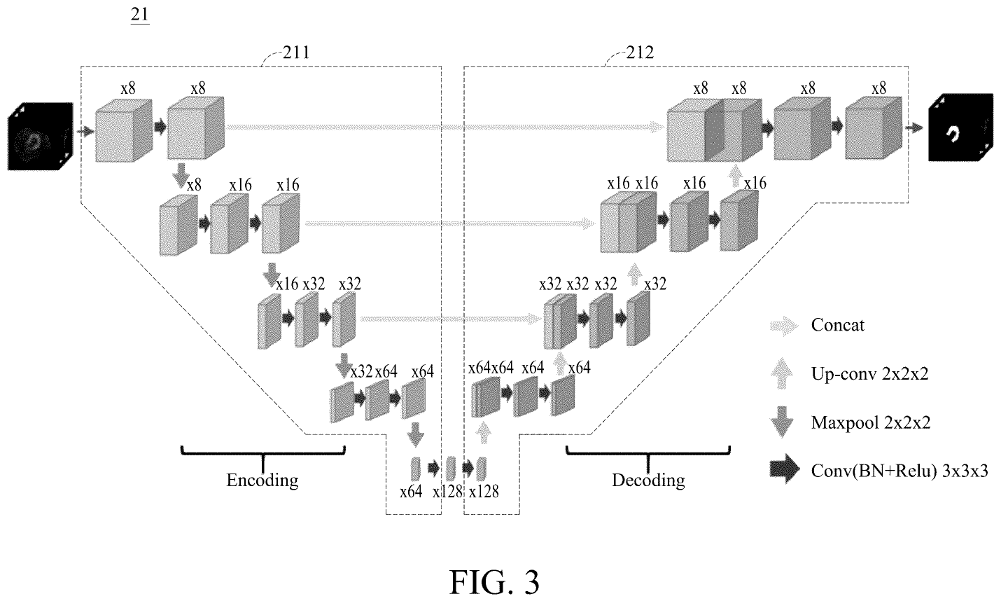

In one embodiment, the U-net model used for the LV myocardial segmentation is a 3D U-net model 21, which architecture is shown in FIG. 3. As seen in FIG. 3, the left arm 211 of this 3D U-net model 21 is configured to encode the 3D images into high-dimension feature maps by using convolution layers and activation layers, where size of feature map output from said convolution layers and activation layers in each step is down-sampled into half by the max-pooling layer following thereafter. The down-sampling process within the left arm 211 is configured to double receptive field of the convolution layer and activation layers without changing convolution kernel size. The right arm 212 of this 3D U-net model 21 is configured to up-sample the feature map at the end of the left arm 211 stepwise, combine (concat) the information provided from the left arm 211, and output the predicted myocardial segmentation (LV myocardium images). During study, the 3D U-net model 21 is trained using a manually segmented dataset of LV myocardium images from 500 subjects, and is trained until maximum of Dice's coefficient between prediction result and manual segmentation is reached. However, skilled artisan in this technical field should understand that the training process for the 3D U-net model 21 is not meant to limit the scope of this disclosure, and can be trained by other applicable methods.

In one embodiment, the rigid registration is configured to align LV myocardium images from the 3D U-net model 21 to the myocardium template. In the embodiments described herein, a rigid registration algorithm is first conducted to allow moving, rotating and resizing of the LV myocardium images to match with the myocardium template. Then, the rigid registration process is iteratively performed with a gradient optimizer to minimize the intensity difference between the LV myocardium images. The rigid registration performed herein is beneficial for making LV myocardium flattening in a later process space-invariant, which will be discussed later.

In one embodiment, normalization of the LV myocardium images is performed under the assumption that at least 20% of myocardium during MPI is normally perfused. From here, the image values of the LV myocardium images are divided by average of greatest 20% of voxel values of segmented myocardium thereof, making the average of normally perfused myocardium to have scaled value of 100%.

### Creation of Gender Specific NDBs and TPD Quantification

To test the performance of the system 1 of the present disclosure compared to that of existing techniques, a gender specific NDB is built and a 3D TPD (TPD_{3D}) quantification is also conducted on the LV myocardium images of the enrolled 1861 subjects based on the gender specific NDB for prediction of obstructive CAD to serve as a control group for development of system 1 of the present disclosure.

The building process for the gender specific NDB is described as follows. First, a NDB is initially built by collecting MPI image sets from a group of subjects (e.g., 15 men and 15 women being additionally collected) with normal MPI (i.e., both summed stress score and SRS less than 4) and angiographically documented patent coronary arteries. These images were further imported into the database generator of the commercially available Quantitative Perfusion SPECT software (QPS, Cedar-Sinai Medical Center, USA). Each subject from either parameterization group or validation group was processed by the QPS software without manual adjustment of LV contour. The stress TPD value as well as the TPD values in each vessel territories were automatically calculated to serve as baseline for comparison.

Since the embodiment of the present disclosure directly processes 3D SPECT images rather than polar map, a 3D version of TPD (TPD_{3D}) in addition to the conventional TPD quantification was used for better comparison. The images from NDB were automatically preprocessed using the above-mentioned steps. Then, the 3D NDB was established by calculating the mean and standard deviation (SD) of each voxel in a segmented myocardial region (e.g., segmentation may be performed using the 3D U-net model 21) from 3D image of MPI image sets of the subjects. Next, each subject from either group was compared to this 3D NDB, and each voxel in segmented myocardial region of the 3D images is given a score using the following definition:

$$score ( x ) = \{ \begin{matrix}0 , x > mean - S D \\ 1 , x \leq mean - S D and x > mean - 2 S D \\ 2 , x \leq mean - 2 S D and x > mean - 2.25 S D \\ 3 , x \leq mean - 2.25 S D and x > mean - 2. S D \\ 4 , x \leq mean - 2.5 S D\end{matrix} \qquad equation ( 1 )$$

Then, the voxel score values of each 3D images are summed together and normalized into a 3D total perfusion deficit (TPD_{3D}):

$$T P {D}_{3 D} = \frac{{\sum }_{x ∈ myocardial voxels} score ( x )}{4 \times number of myocardial voxels} \times 1 \; 00 \% \qquad equation ( 2 )$$

In addition to patient-based TPD3D values, TPD3D values in each vessel territories, which defined by the American Heart Association 17-segment myocardial model, were calculated.

### Generation of 3D Blackout Images

Another objective of system 1 of the present disclosure is to eliminate the requirement for PM during prediction of obstructive CAD. For this purpose, 3D blackout images are generated from the LV myocardium images of the enrolled 1861 subjects based on the gender specific NDB. Said 3D blackout images are treated as added value for the deep learning module 40 when the LV myocardium images of the enrolled 1861 subjects are fed therein for prediction of obstructive CAD.

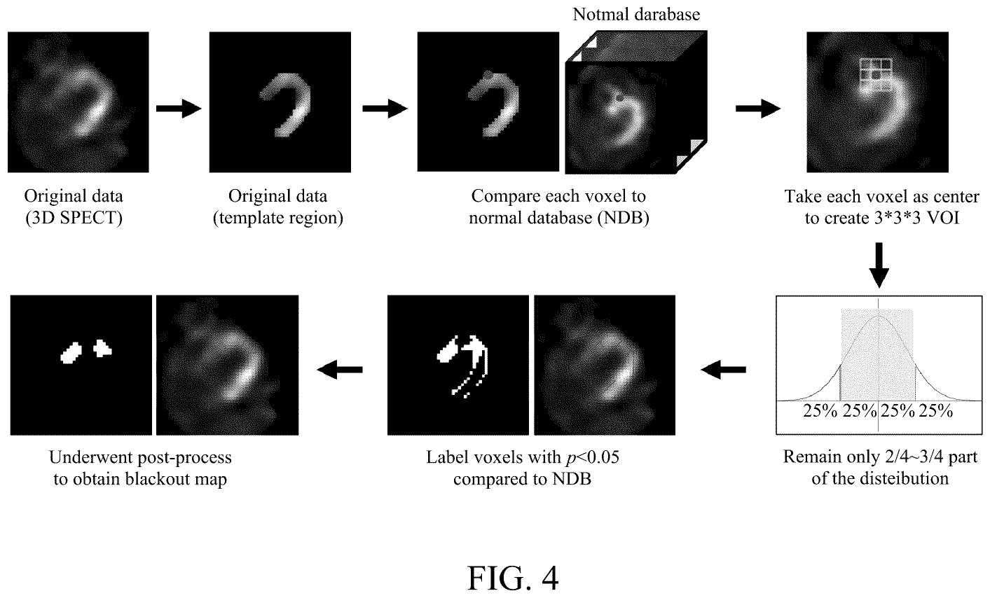

During study, spatially matched 3D blackout image corresponding to the LV myocardium images of the enrolled 1861 subjects are generated to enhance the regions of hypoperfusion in the LV myocardium images. The process for generation of 3D blackout images is illustrated in FIG. 4, where a one-tailed Student's t-test is used to compare each of myocardial voxel value within the LV myocardium images to the gender specific NDB including neighboring voxels, the voxels with a p-value of less than 0.05 were marked as significant, a level-set algorithm is used to segment normally perfused myocardium from the LV myocardium images, the small marked regions that overlapped with the normal myocardium were removed, and the backout image is generated by forcedly setting the voxel values in marked region to be zero.

### LV Myocardium Flattening

CNNs are known to be invariant to image translation but not to image rotation, given that convolution operations of CNNs are applied like sliding window, and the effects of translation is reduced by pooling operations. However, this is problematic in processing 3D MPI. For example, a convolution kernel of a CNN that excels in capturing an activity pattern in anterior wall during processing of MPI might not be applied well to septum and lateral wall. The problem can be partially solved by introducing manual rotation into training dataset of CNNs. However, augmented rotated images may break the empirical territory of epicardial vessels, thus influence performance in predicting obstructive CAD in individual vessels.

To avoid the problems described above, image transformation is utilized in flattening module 30 of this disclosure, which is to introduce spherical coordinate transformation to LV myocardium images. Spherical coordinate transformation is similar to polar map (PM) process in the sense that it flattens the myocardium, but much suitable for obstructive CAD prediction in that it preserves 3D information within the LV myocardium images. With this additional benefit in mind, the flattening module 30 is configured to generate flattened images by resample the LV myocardium images (the data of which is in Cartesian coordinate system and is space-invariant due to rigid registration performed by the pre-processing module 20) generated from the pre-processing module 20 into 3D spherical coordinate system, so as to preserve the neighborhood relationship between adjacent myocardium for the deep learning module 40 during prediction of obstructive CAD.

In the embodiments described herein, said resampling performed by the flattening module 30 is conducted by resampling the LV myocardium images using the spherical coordinate system with an origin located at center of LV chamber therein, hence transforming the LV myocardium images into a 22×46×18 matrix based on the Cartesian coordinate system.

### Network Structure and Training

During study, it is proven that various CNNs trained specifically for predicting obstructive CAD in vessels do not perform well in identifying subjects without any obstructive disease (i.e., subjects having patent vessels) due to attenuation artifacts common in MPI. As such, it is beneficial to train an additional model (i.e., the patent prediction network 42) to identify attenuation artifacts in MPIs (in this case, LV myocardium images) for predicting subjects without any obstructive disease at patient-level. In this way, a probability of a subject having patent coronary arteries predicted by the patent prediction network 42 may be incorporated into the loss function of the disease prediction network 41 (which is trained to predict individual probability of obstructive CAD in LAD, LCX and RCA), thus improve prediction ability of the deep learning module 40.

As mentioned, the deep learning module 40 is consisted of the disease prediction network 41 and the patent prediction network 42. In the embodiment where both the disease prediction network 41 and the patent prediction network 42 are CNNs, the disease prediction network 41 is configured to predict probability of obstructive CAD in LAD, LCX and RCA separately, and the patent prediction network 42 is configured to predict probability of subjects with patent coronary arteries.

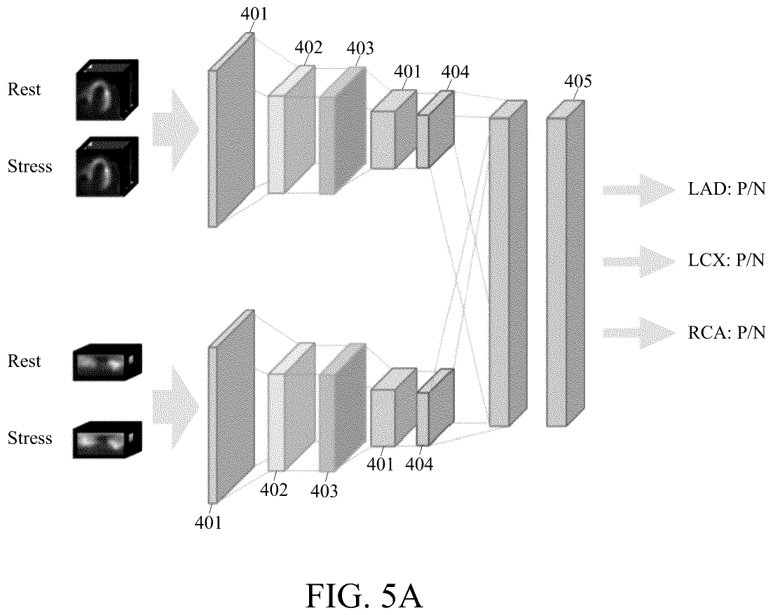

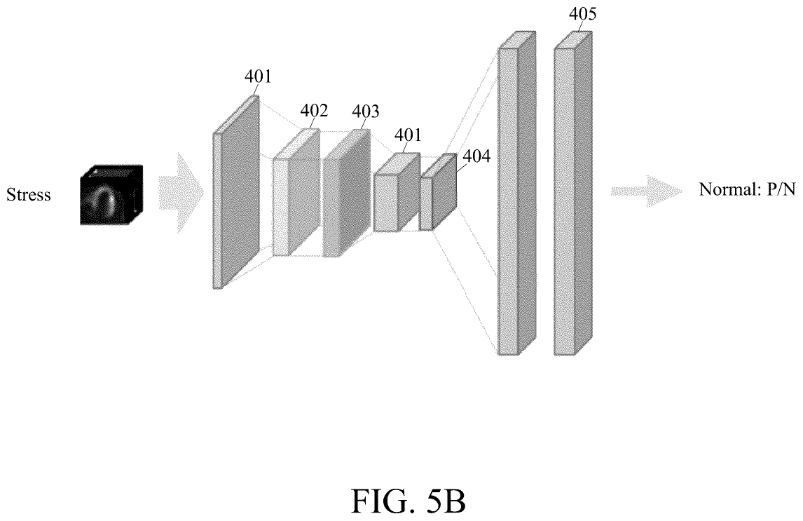

Referring to FIGS. 5A and 5B, structures of the disease prediction network 41 and the patent prediction network 42 of the deep learning module 40 in accordance to the present disclosure is shown, where both the disease prediction network 41 and the patent prediction network 42 may be viewed as two parts: a first part being configured for feature extraction; and a second part being configured for using feature extracted in prediction of obstructive CAD in LAD, LCX and RCA (for the disease prediction network 41) or patent coronary arteries (for the patent prediction network 42). Specifically, the first part is consisted of convolution layers 401, spatial reduction blocks 402, residual convolution blocks 403, and max-pooling layers 404, where each convolution layers 401 is followed by a batch normalization layer and a rectified linear unit activation function (not shown), and the spatial reduction blocks 402 and the residual convolution blocks 403 are configured to enable integration of information from different receptive fields. On the other hand, the second part is consisted of one or more fully-connected layers 405 (preferably three layers), each of which is followed by a sigmoid activation function (not shown).

On a closer look at FIGS. 5A and 5B, the structure of the disease prediction network 41 is different from that of the patent prediction network 42 in that: the disease prediction network 41 is constructed with two channels of the first part, so as to take LV myocardium images in both post-stress form and rest form from the pre-processing module 20 and the corresponding flattened images (also in post-stress form and rest form) from the flattening module 30 as input for prediction of obstructive CAD in LAD, LCX and RCA of the subjects respectively, while the patent prediction network 42 only requires one channel of the first part to take LV myocardium images in post-stress form as input for prediction of patent coronary arteries of the subjects.

In some embodiments, the disease prediction network 41 and the patent prediction network 42 and their training programs are implemented in Python with Keras framework, where their training process is conducted on a workstation with graphic processing unit (GPU, Titan RTX, NVIDIA, California, United States) acceleration. However, it should be noted that the training environment of the disease prediction network 41 and the patent prediction network 42 is not meant to restrict scope of the present disclosure, and can be realized in other applicable environments.

In at least one of the embodiment, the angiographic result was used to train patent prediction network 42 (e.g., the patent CNN) in the first step of training. In the second step, the information learned by the patent prediction network 42 was integrated to guide the training of the disease prediction network 41 (e.g., the disease CNN). The training result of the deep learning module 40 is determined by a compound loss function of two cross-entropy based on the performances of the disease prediction network 41 and the patent prediction network 42:

$$Loss = - {\sum }_{c = 1}^{3} {\sum }_{i = 1}^{n} {y}_{c , i} {log}_{2} ( {p}_{c , i} \times ( 1 - {N}_{{p}_{i}} ) ) , \qquad equation ( 3 )$$

where n represents number of training cases, y_{c,i} represents label of whether vessel c of case i is obstructive, p_{c,i} represents predicted probability (network output) of obstructive vessel c of case i, and Nₚ_{ᵢ} represents predicted probability (network output) of patent coronary arteries of case i.

The deep learning module 40 is trained to minimize the above loss function in training dataset (i.e., LV myocardium images and flattened images corresponding to MPI image sets related to internal parameterization group), where the training process is set to stop when the loss function reaches 1300 epochs. Then, the deep learning module 40 may be put into practical use for predicting obstructive CAD.

### Validation

To evaluate ability of generalization of the system 1 during practical use, two methods are utilized to validate the system 1 during and after development, namely, cross-validation and external validation. During development, a 10-fold cross-validation is applied to make full use of each subject in the internal parameterization group and to prevent overfitting, where said 10-fold cross validation comprises the steps of: partitioning LV myocardium images related to the internal parameterization group into 10 mutually exclusive subsets while disease prevalence of each vessel is balanced: repeating training process of the deep learning module 40 10 times, where each round of training process uses a different combination of 9 subsets as training data and remaining 1 subset as validation data; and joining the 10 validation results from the 10 training process together as representation of performance of trained deep learning module 40. After development, external validation is applied to further evaluate the robustness of the deep learning module 40, where said external validation comprises the steps of: re-train the deep learning module 40 using all LV myocardium images related to internal parameterization group as training data; and validate the deep learning module 40 using LV myocardium images related to the external validation group as validation data.

### Statistical Analysis

Analysis on performance of the system 1 of the present disclosure is deployed based on various aspects. For example, continuous baseline characteristics derived from performance of system 1 are compared using Wilcoxon rank sum test, and categorical baseline characteristics derived from performance of system 1 are compared using Pearson's Chi-square test. Further, receiver-operating characteristic (ROC) analysis is used to evaluate performance of the system 1 in predicting obstructive CAD, where a DeLong test is applied to compare difference between area under ROC curves (AUC) of prediction with (representing performance of purposed system 1 using NDB-derived information) and without (representing performance of purposed system 1) the 3D blackout images. In the embodiment where statistical analysis on performance of system 1 is carried out using R software, a p-value of less than 0.01 is considered to be statistically significant.

### Results

First, the continuous and categorical baseline characteristics of the enrolled 1861 subjects for development of the system 1 of the present disclosure are shown in Table 1 below, where a significantly low prevalence of dyslipidemia and borderline higher prevalence of hypertension are shown in external validation group (see column represented as “EVG”), and a higher proportion of subjects referred for treadmill exercise stress is shown in external validation group compared to that in internal parameterization group (see column represented as “IPG”).

TABLE 1
Baseline characteristics of enrolled subjects

| | | | Group | | |
|---|---|---|---|---|---|
| | All | | IPG | EVG | |
| Characteristics | n = 1861 | | n = 928 | n = 933 | p-value |
| Gender | | | | | 0.08 |
| Male | 1407 | (76%) | 685 (74%) | 722 (77%) | |
| Female | 454 | (24%) | 243 (26%) | 211 (23%) | |
| Age (years) | 64.1 ± 11.8 | | 64.7 ± 11.9 | 63.5 ± 11.6 | 0.04 |
| BMI (kg/m²) | 26.1 ± 3.9 | | 26.3 ± 4.0 | 26.1 ± 3.8 | 0.21 |
| Risk factors | | | | | |
| Hypertension | 1315 | (71%) | 681 (73%) | 634 (68%) | 0.01 |
| Diabetes mellitus | 634 | (34%) | 337 (36%) | 297 (32%) | 0.05 |
| Dyslipidemia | 962 | (52%) | 508 (55%) | 454 (49%) | <0.01 |
| Current smoker | 387 | (21%) | 203 (22%) | 184 (20%) | 0.28 |
| Stress type | | | | | <0.01 |
| Treadmill | 426 | (24%) | 188 (20%) | 238 (26%) | |
| Pharmacologic | 1435 | (76%) | 740 (80%) | 695 (74%) | |

| | | | | |
|---|---|---|---|---|
| Stress TPD | 12.2 ± 13.9 | 14.1 ± 14.7 | 10.3 ± 12.8 | <0.01 |

Continuous parameters are represented as mean ± standard deviation, and categorical parameters are expressed as count and percentage.
IPG: internal parameterization group
EVG: external validation group
BMI: body mass index;
TPD: total perfusion deficit

Further, the comparison of patient characteristics of the enrolled 1861 subjects with or without obstructive disease are shown in Table 2 below. There was significantly older age, higher proportion of male gender, and higher prevalence of hypertension, diabetes mellitus, and dyslipidemia in patients with obstructive disease.

TABLE 2
Comparison of patient characteristics
with or without obstructive disease

| | Obstructive disease | | |
|---|---|---|---|
| | Yes | No | |
| Characteristics | n = 1122 | n = 739 | p-value |
| Gender | | | <0.01 |
| Male | 886 (79%) | 521 (71%) | |
| Female | 236 (21%) | 218 (23%) | |
| Age (years) | 66.0 ± 11.0 | 61.1 ± 12.3 | <0.01 |
| BMI (kg/m²) | 26.1 ± 3.8 | 26.4 ± 4.1 | 0.53 |
| Risk factors | | | |
| Hypertension | 837 (75%) | 478 (65%) | <0.01 |
| Diabetes ellitus | 468 (42%) | 166 (22%) | <0.01 |
| Dyslipidemia | 620 (55%) | 342 (46%) | <0.01 |
| Current smoker | 328 (21%) | 149 (20%) | 0.73 |
| Stress type | | | 0.02 |
| Treadmill | 234 (21%) | 189 (26%) | |
| Pharmacologic | 888 (79%) | 550 (74%) | |
| Stress TPD | 16.6 ± 15.1 | 5.4 ± 8.2 | <0.01 |

Continuous parameters are represented as mean ± standard deviation, and categorical parameters are expressed as count and percentage.
BMI: body mass index;
TPD: total perfusion deficit

In addition, the angiographic characteristics of the enrolled 1861 subjects for development of the system 1 of the present disclosure are shown in Table 3 below, where an average one month (about 30 days) interval between MPI and ICA are recorded among the enrolled subjects, prevalence of obstructive CAD is 60% in the enrolled subjects, and no significant difference of angiographic characteristics is shown between internal parameterization group (see column represented as “IPG”) and external validation group (see column represented as “EVG”).

TABLE 3
Angiographic characteristics of enrolled subjects

| | | Group | | |
|---|---|---|---|---|
| | All | IPG | EVG | |
| Characteristics | n = 1861 | n = 928 | n = 933 | p-value |
| Interval to ICA (days) | 32 ± 21 | 31 ± 20 | 32 ± 21 | 0.82 |
| Patent arteries | 314 (17%) | 142 (15%) | 172 (18%) | 0.08 |
| Non-obstructive | 429 (23%) | 207 (22%) | 222 (24%) | 0.48 |
| 1-vessel-disease | 487 (26%) | 254 (27%) | 233 (25%) | 0.26 |
| 2-vessel-disease | 364 (20%) | 189 (20%) | 175 (18%) | 0.41 |
| 3-vessel-disease | 267 (14%) | 136 (15%) | 131 (14%) | 0.76 |
| Obstructive LAD disease | 774 (42%) | 400 (43%) | 374 (40%) | 0.20 |
| Obstructive LCX disease | 602 (32%) | 319 (34%) | 283 (30%) | 0.07 |
| Obstructive RCA disease | 640 (34%) | 321 (35%) | 319 (34%) | 0.89 |

ICA: invasive coronary angiography
LAD: left anterior descending artery
LCX: left circumflex artery
RCA: right coronary artery

In the embodiment, each element of the system 1 is configured in the sense that all MPI image sets related to the enrolled 1861 subjects are fully-automatically pre-processed and visually inspected for the purpose of obstructive CAD prediction, meaning no manual adjustment is required for operation of the system 1 of the present disclosure.

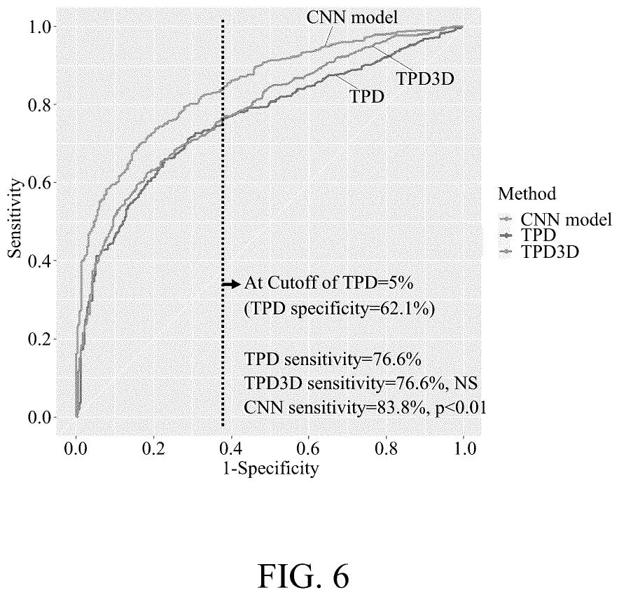

The performance of the system 1 of the present disclosure based on the enrolled 1861 subjects is shown in FIGS. 6, 7, 8A, and 8B.

Patient-based analysis of the performance of system 1 (CNN model in the embodiment) is shown in FIG. 6. AUC (0.844) of prediction on obstructive CAD of subjects (internal parameterization group) from the 10-fold cross-validation of the system 1 of the present disclosure is shown to be higher than either the AUC (0.795) of prediction on obstructive CAD of subjects using the TPD (p-value <0.01) or the AUC (0.781) of prediction on obstructive CAD of subjects using the TPD_{3D} (p-value <0.01). The performance of TPD_{3D} was also mildly better than TPD (p=0.03). Also, the AUC (0.841) of prediction on obstructive CAD of subjects from the system 1 with addition of 3D blackout image is no different than the AUC (0.844) of prediction on obstructive CAD of subjects without usage of 3D blackout image (p-value=0.72). In addition, using empirical 5% TPD cutoff, the sensitivity and specificity of conventional TPD were 76.6% and 62.1%, respectively. While maintaining the same level of specificity, the sensitivity of the TPD_{3D} method did not change (also 76.6%). On the contrary, the sensitivity of the system 1 of the present disclosure was significantly improved (83.8% vs 76.6%, p<0.01, with cutoff at 33% probability). The results shown in FIG. 6 proves that the usage of 3D information in MPI contains more useful information in prediction of obstructive CAD compared to NDB-derived abnormal areas.

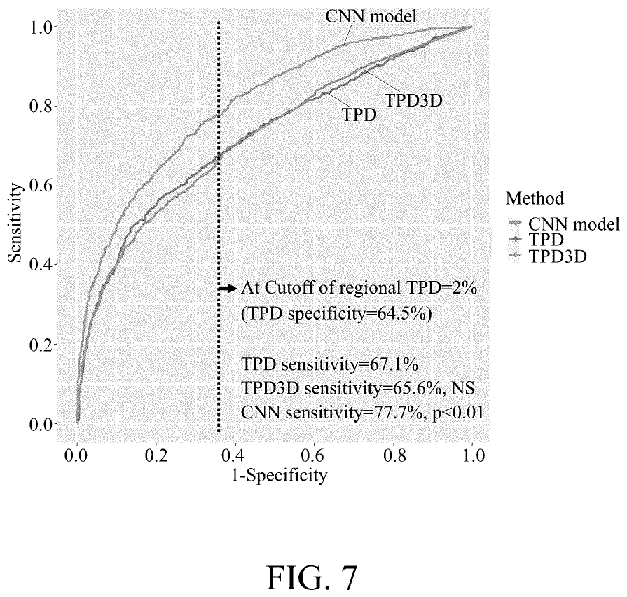

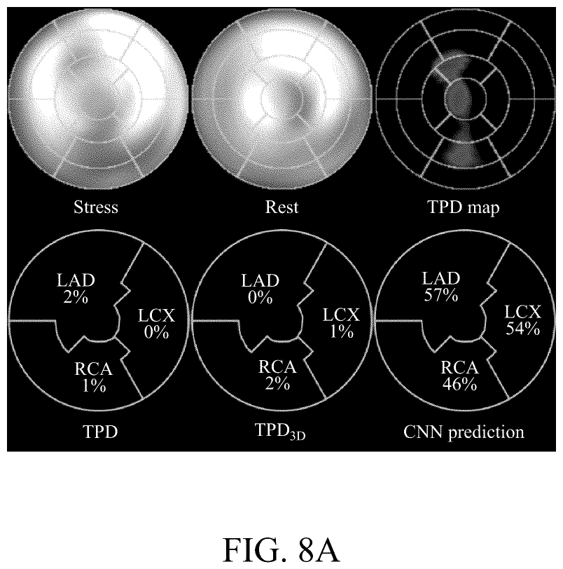

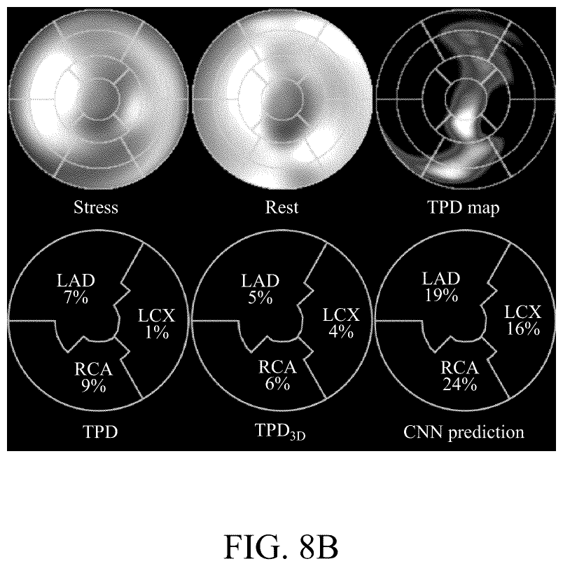

Vessel-based analysis of the performance of system (CNN model in the embodiment) is shown FIG. 7. AUCs of predictions on obstructive CAD of LAD (0.81), LCX (0.81), and RCA (0.80) of subjects (internal parameterization group) from the 10-fold cross-validation of the system 1 of the present disclosure are shown to be significantly higher than either AUCs of predictions on obstructive CAD of LAD (0.72), LCX (0.73), and RCA (0.70) using the TPD (all p-values <0.01) or AUCs of predictions on obstructive CAD of LAD (0.73), LCX (0.74) and RCA (0.73) using the TPD_{3D}(all p-values <0.01). Furthermore, AUCs of predictions on obstructive CAD of LAD, LCX and RCA from the system 1 with addition of 3D blackout images are no different than the AUCs of predictions on obstructive CAD of LAD, LCX and RCA without usage of quantitative blackout image (all p-values >0.05). In addition, using empirical 2% regional TPD cutoff, the sensitivity and specificity of conventional TPD were 67.1% and 64.5%, respectively. While maintaining the same level of specificity, the sensitivity of the system 1 of the present disclosure was also significantly improved (77.7% vs 67.1%, p<0.01). Representative cases were shown in FIGS. 8A and 8B. FIG. 8A shows that there was only minor deficit quantified by TPD: however, system 1 of the present disclosure correctly predicted multi-vessel disease (cutoff at 33% probability). FIG. 8B shows that the images showed defects which were not typical for either vessel territory, and the system 1 of the present disclosure correctly predicted low probability (<33%) of obstructive disease.

In addition, the system 1 of the present disclosure after re-trained with external validation group is also tested, where AUC (0.819) from the patient-based analysis and AUC (0.783) from the vessel-based analysis are not significantly different from the AUCs of 10-fold cross-validation of the system 1 (p-value=0.17 and 0.12, respectively). Therefore, it is implied that the development and training for the system 1 of the present disclosure is not overfitting and the prediction performance is consistent across subjects from different time periods.

### Discussions

The system 1 of the present disclosure proposed a 3D DL technique for prediction of obstructive CAD from MPI. The proposed technique does not require manual correction of LV contour, traditional process of PM generation, and quantitative comparison with NDB. With validation on prediction result from the enrolled 1861 subjects with reference standard of their ICA results, the proposed technique outperformed TPD_{3D} in both patient-based analysis and vessel-based analysis.

### CONCLUSIONS

In this disclosure, the 3D DL technique described is proven to be beneficial in predicting obstructive CAD from CZT myocardial perfusion SPECT, given that it does not require a polar map, manual correction or NDB derived quantification, and the performance in prediction of obstructive CAD has outperformed traditional TPD quantification.

The present disclosure has been described with exemplary embodiments to illustrate the principles, features, and efficacies of the present disclosure, but not intend to limit the implementation scope of the present disclosure. The present disclosure without departing from the spirit and scope of the premise can make various changes and modifications by a person skilled in the art. However, any equivalent change and modification accomplished according to the disclosure of the present disclosure should be considered as being covered in the scope of the present disclosure. The scope of the disclosure should be defined by the appended claims.

What is claimed is:

1. A system for predicting obstructive coronary artery disease of a subject in need thereof, comprising:
a pre-processing module, configured to pre-process a myocardial perfusion imaging (MPI) image set of the subject into left ventricular myocardium images of post-stress form and rest form, wherein the MPI image set comprises 3D images of the post-stress form and the rest form:
a flattening module, configured to resample the left ventricular myocardium images into flattened images of the post-stress form and the rest form, wherein the flattened images comprises data in a 3D spherical coordinate system; and
a deep learning module, configured to take the left ventricular myocardium images and the flattened images as input for predicting of the obstructive coronary artery disease of the subject.

2. The system of claim 1, wherein the pre-processing of the pre-processing module comprises steps of:
performing a left ventricular myocardium segmentation on the 3D images via a U-net model to obtain the left ventricular myocardium images; and
performing a rigid registration to align each of the left ventricular myocardium images to a myocardium template according to a left ventricular myocardium segmented therefrom.

3. The system of claim 1, wherein the deep learning module comprises a disease prediction network and a patent prediction network, wherein the disease prediction network is configured to predict probabilities of obstructive coronary artery disease in left anterior descending, left circumflex and right coronary artery, respectively, for the subject, and wherein the patent prediction network is configured to predict a probability of patent coronary arteries for the subject.

4. The system of claim 3, wherein the disease prediction network is configured to take the left ventricular myocardium images and the flattened images in both the post-stress form and the rest form as input for prediction, and wherein the patent prediction network is configured to take the left ventricular myocardium image in the post-stress form as input for prediction.

5. The system of claim 3, the disease prediction network and the patent prediction network are convolution neural networks.

6. A method for predicting obstructive coronary artery disease of a subject in need thereof, comprising:
having a pre-processing module pre-process a myocardial perfusion imaging (MPI) image set of the subject into left ventricular myocardium images of post-stress form and rest form, wherein the MPI image set comprises 3D images of the post-stress form and the rest form:
having a flattening module resample the left ventricular myocardium images into flattened images of the post-stress form and the rest form, wherein the flattened images comprises data in 3D spherical coordinate system; and
having a deep learning module take the left ventricular myocardium images and the flattened images as input for predicting the obstructive coronary artery disease of the subject.

7. The method of claim 6, wherein the pre-process of the pre-processing module comprises:
performing a left ventricular myocardium segmentation on the 3D images via a U-net model to obtain the left ventricular myocardium images; and
performing a rigid registration to align each of the left ventricular myocardium images to a myocardium template according to a left ventricular myocardium segmented therefrom.

8. The method of claim 6, wherein the deep learning module comprises a disease prediction network and a patent prediction network, wherein the disease prediction network is configured to predict probabilities of obstructive coronary artery disease in left anterior descending, left circumflex and right coronary artery, respectively, for the subject, and wherein the patent prediction network is configured to predict a probability of patent coronary arteries for the subject.

9. The method of claim 8, wherein the disease prediction network is configured to take the left ventricular myocardium images and the flattened images in both the post-stress form and the rest form as input for prediction, and wherein the patent prediction network is configured to take the left ventricular myocardium image in the post-stress form as input for prediction.

10. The method of claim 8, the disease prediction network and the patent prediction network are convolution neural networks.
